(19) United States
(12) Patent Application Publication
(10) Pub. No.: US 2006/0134657 A1
(43) Pub. Date: Jun. 22, 2006

(54) Nanoscale electronic detection system and methods for their manufacture

(75) Inventors: Dalibor Hodko, Poway, CA (US); Daniel Smolko, Jamul, CA (US); Stuart F. Duffy, San Diego, CA (US)

Correspondence Address: O'MELVENY & MYERS LLP, 610 NEWPORT CENTER DRIVE, 17TH FLOOR, NEWPORT BEACH, CA, 92660, US

(73) Assignee: NANOGEN, INC., San Diego, CA (US)

(21) Appl. No.: 11/140,555

(22) Filed: May 27, 2005

Related U.S. Application Data
(60) Provisional application No. 60/575,445, filed on May 28, 2004.

(51) Int. Cl. C12Q 1/68

(52) U.S. Cl. 435/6; 977/924

(57) ABSTRACT

A new, extremely sensitive, and rapid electronic detection method for direct detection of hybridized genomic targets to specific probes on the microarray is proposed. The method consists of fast electronic accumulation of the DNA target on a particular electrode site at the micro-electrode array, sequential electronic hybridization of oligonucleotide labeled metallic (nano)particles on the target DNA and monitoring the electrochemical AC impedance changes at the electrode site. The method is enhanced by electroplating over the DNA target which serves as the metallization template and over the particles which provide seeds for rapid electroplating. The AC impedance changes are monitored during the electroplating over the DNA target and between the array electrodes sites. The signal in the absence and presence of the target DNA is a difference between “no connection” and a “short” between the array electrodes

### RELATED APPLICATIONS

This application claims priority to U.S. Provisional Application Ser. No. 60/575,445, filed May 28, 2004, entitled “Nanoscale Electronic Detection System”, and is incorporated herein by reference as if fully set forth herein.

### FIELD OF THE INVENTION

This invention relates to microscale and nanoscale electronic systems and methods for their manufacture. More particularly, the apparatus and methods relate to detectors, especially single sequence detection systems.

### BACKGROUND OF THE INVENTION

Sequencing of the Human Genome induced a new knowledge in understanding the correlations between the DNA structure, gene functions, efficiency of targeted therapeutics as well as occurrence and development of a variety of genetic and infectious diseases. Molecular diagnostics based on DNA revealed mechanisms and advancement of numerous dangerous diseases including cancer, HIV, cystic fibrosis, heart and lung diseases, emerging infectious diseases, to name a few. Although there are several examples where rapid and sensitive DNA analysis is needed, e.g., infectious diseases and biological agent detection, faster and more sensitive DNA detection methods will benefit all areas of human health. Recent threats of bioterrorism attacks as well as the appearance of emerging infectious diseases prompted urgent development of new more sensitive, simple and rapid point-of-use or point-of-care equipment for the detection and identification of pathogens in various medical or environmental samples. Currently, no platforms exist for differentiation between common pulmonary infections and emerging infectious pathogens to offer rapid screening in emergency rooms or in doctor's offices. The DNA microarray platform allows a highly multiplexed recognition of a large number of characteristic genes.

However, the sensitivity is often not satisfactory for detecting a small number of pathogens or cancer cells in a limited sample volume. The DNA based technology still relies on the PCR, polymerase chain reaction amplification or similar molecular amplification techniques to enhance the concentration of DNA. These techniques are time consuming, usually requiring from 30 minutes to 2 hours to achieve a satisfactory amplification. There is an urgent need for new DNA technologies that will require no lengthy molecular amplifications and will be capable of directly detecting specific DNA sequences. Natural DNA hybridization process offers such specificity; however, most standard DNA microarray technologies rely on passive diffusion and hybridization of the target DNA to the probes on the microarray chip which usually takes several hours to accomplish. Electronically-driven microarray technology (See Reference(s) 1-8) (e.g., www.nanogen.com, provides fast transport of DNA sequences (less than one minute) to a specific location on the chip. The detection is accomplished with fluorophore reporters and laser based fluorescence detection (See Reference(s) 9-14). The technology utilizes PCR or strand displacement (SDA) amplified DNA as the target sample which consumes time and renders these methods incompatible for use in emergent situations where the desirable total analysis time is less than 20 minutes.

DNA microarray technology has critical advantages compared to other methods for DNA based analysis of single nucleotide polymorphisms (SNPs), short tandem repeats (STRs) for human identification or for the detection of viruses and pathogens because of its inherent possibilities for multiplexed detection on large number of array spots. Recently, a number of methods for the detection of pathogens or viruses have been developed (See Reference(s) 15-19), however major disadvantages for their use as practical portable systems in the field are that they do not satisfy the requirement of highly multiplexed detection. Often detection limits, weight limits or accuracy, or the need for skilled personnel to operate the instrument, renders those instruments to be non-compliant with the desirable specifications. A portable hospital or clinical lab instrument for DNA based molecular diagnostics should be light-weight (less than around 20-30 lbs), capable of specific detection of series of genes, characteristic for particular SNPs or pathogens (panels with 20 up to 100 characteristic genes are desirable) with analysis time less than 0.5 to one hour and detection limits approaching only few copies of DNA or 10-100 cfu/ml.

In the last decade, the development of microarrays has greatly expanded our analytical capabilities for protein and DNA analysis (See Reference(s) 20). Many novel techniques now allow us to simultaneously analyze thousands of DNA sequences in microliter volumes at the picomolar level of sensitivity. Examples include Affymetrix's GeneChip™ (See Reference(s) 21-23), Nanosphere's (See Reference(s) 24) gold nanoparticle technology and Nanogen's electronically active Nanochip® (See Reference(s) 25) technologies. Assays have been developed for gene expression analysis (See Reference(s) 26), forensics (See Reference(s) 27), SNP (See Reference(s) 28) analysis and a host of other novel assay formats.

Competitive DNA based portable systems are developed today mostly for the emergent applications such as the detection of biological warfare agents or pathogens. These include Idaho Technology's Ruggedized Advanced Pathogen Identification Device (R.A.P.I.D System) and Rapid Cycler systems (See Reference(s) 29), Autonomous Pathogen Detection System (APDS) developed at LLNL (See Reference(s) 30-31), and Cepheid's Smart Cycler system (See Reference(s) 32-34) that are capable of integrating on-chip lysis of microorganisms, amplification of their characteristic DNA through the polymerase chain reaction and fluorescence detection of DNA. Although many of these systems offer elegant solutions to detection of a smaller number of agents, the number of optical channels installed limits their application when a larger number of agents or genes needs to be detected. Compared to those technologies, the microarray platform practically does not pose a limit to multiplexed detection of large number of pathogens as well as their characterization by multiple genes. Today no portable point-of-care microarray based DNA analysis system has been developed for commercial use.

Recently, the Nanochip® microarray technology has developed a portable electronic microarray system which accommodates an electrode array with 400 sites and uses fluorescence based detection for the detection of addressed DNA targets. Assays for Factor II and V, SNPs for human identification based on mitochondrial DNA, as well as assays for emerging infectious disease and biological warfare pathogens have been developed.

All of the above techniques utilize PCR or similar molecular amplification techniques to amplify the DNA target in the sample. This proposal initiates the development of a new direct electronic DNA detection technique which will not need PCR or other long-term amplification methods to amplify the DNA concentration in the sample. The method will provide a new microarray-based platform for extremely rapid DNA analysis which will be highly sensitive and specific for a particular set of targeted genes. The electronics-based detection technique will allow design of a small, portable, potentially hand-held microarray instrument and will not need more complex and field-sensitive optical detection system consisting of sensitive lasers, lenses and other optical components.

The intrinsic conductivity of bare DNA is too low to allow its utilization as a molecular wire or to directly measure its presence through simple conductance measurements between two electrode sensors (See Reference(s) 35-36). The localization and binding of few target DNA molecules between the electrodes or on the substrate at a desired location is extremely slow because this step is controlled by a slow difflusion process. If the concentration of the analyte is only a few molecules of DNA the passive process of capturing DNA has very low statistic probability. The proposed technology easily overcomes these problems by directional and fast electrophoretic transport of DNA targets toward the electrode array sites.

Several different DNA metallization techniques have been reported (See Reference(s) 37) utilizing various metals, including silver (See Reference(s) 38), palladium (See Reference(s) 39), and platinum (See Reference(s) 40). In general, those methods are based on electro-less plating processes which usually consist of two steps. Metallic clusters are first formed on the DNA, and then used as nucleation sites for selective metal deposition in a subsequent metal reduction process until a continuous metallization of the DNA molecule is obtained. The formation of metallic nucleation centers relies on binding of metal ions or complexes to the DNA and their subsequent reduction to form metallic clusters, or on binding of small metallic particles to the DNA.

These metallization techniques suffer from several drawbacks. First, these metallization processes are very slow, particularly if based on particle binding to DNA. They are uniform over the entire DNA scaffold, thus non-specific as well as yield to a highly non-specific deposition of metallic ions or metallic particles on the substrate at locations where no DNA is present causing a high level of false positive signals. More importantly, electro-less metallization processes destroy the recognition properties of the DNA, thus preventing any subsequent reporter binding steps through hybridization. A molecular lithography-based method has been recently developed which provides some level of protecting specific sequences of the DNA molecules from the metallization process (See Reference(s) 41). The method involves the metallization of DNA molecules by sequence-specific derivatization with glutaraldehyde, which acts as the localized reducing agent on the DNA. Silver ions are then specifically reduced by the DNA-bound aldehyde groups in the aldehyde-derivatized regions, resulting in the formation of a silver cluster chain along the DNA. An electroless gold deposition process (See Reference(s) 42), catalyzed by the silver clusters is then used to generate continuous DNA-templated gold metallization. The process consists of a number of cumbersome steps which require several reagents that need to be freshly prepared.

A recent review article by J. Wang (See Reference(s) 43) summarized the detection techniques for DNA templated metallization. His group has developed an electrochemical based technique in which deposited silver ions are reduced and subsequently dissolved. The silver ion concentration is then determined using anodic stripping voltammetry (ASV). This technique although highly sensitive for determination of silver ions is prone to high false positive results, because a single silver particle adsorbed at the substrate and not on the DNA molecule will produce a high silver ion ASV signal. Mirkin's group is one of the groups leading the innovation in applying nanoparticle-DNA assemblies to nanofabrication and sensor applications (See Reference(s) 44-47). They have developed an electrical DNA detection method utilizing oligonucleotide ftinctionalized gold nanoparticles and closely-spaced interdigitated microelectrodes (See Reference(s) 48). The oligonucleotide probe was immobilized in the gap between the two microelectrodes. The gold nanoparticles are attached to the DNA target over the oligonucleotide probes. The method involves a subsequent silver deposition which leads to a measurable conductivity signal. The method showed a high sensitivity with a 0.5 pM detection limit. The method proposed in this project differs from this technique in directed and controlled electrophoretic accumulation of both DNA target and oligonucleotide labeled metallic particles as well as introduces electrophoretic amplification of the signal by clustering metallic particles on the template DNA. This assures a high signal-to-noise AC impedance signal measurements of the metallic particles clustering on the metallized DNA through a repeated and/or cyclic electrophoretic process where metallic particle tags yield an amplified signal. The proposed method utilizes a fast directed electroplating of target DNA template as opposed to the sterically non-specific electroless plating. The technical principles of the proposed detection method are summarized in a separate section below.

Nanogen's microarray technology (http://www.nanogen.com) is unique among DNA microarrays due to the use of electrophoretically driven, active transport of the DNA analyte and/or probe molecules at the array. The transport over the array is electronically controlled by connecting the array sites as electrodes. This electronic addressing of biomolecules at the array can accelerate molecular binding on the microchip up to 1,000 times compared to the traditional passive methods. For instance, hybridization on passive microarrays may take up to several hours which is critical when low concentrations of DNA target need to be determined. The most recent version of the Nanochip® is an array of 400 platinum electrodes, 50 pm in diameter, each of which is independently controlled and monitored by circuitry designed into the chip. A thin, hydrogel permeation layer containing co-polymerized streptavidin, covers the surface of the microarray electrodes. The main function of the hydrogel matrix is to provide binding sites for biotin labeled DNA probes; however, it also protects the DNA from the harsh electrochemical environment at the electrode surface. We have taken advantage of the H⁺ generated at the positive electrode to perform electronic hybridization which promotes conditions for efficient DNA hybridization in zwitterionic buffer such as histidine. Nanogen's commercial instruments (Nanochip® System) can use electronic, thermal or chemical techniques, depending on the application, for precise, accurate stringency control. This provides an extremely flexible platform for the assay design allowing several types of multiplexed analyses, e.g. determination of multiple genes in one sample, multiple samples with one gene, or multiple samples with multiple genes. The ability to control individual test sites permits biochemically unrelated molecules to be used simultaneously on the same microchip. In contrast, sites on a conventional DNA array cannot be controlled separately, and all process steps must be performed on an entire array. The commercial system uses fluorescence based detection using fluorophore labeled oligonucleotide probes or reporters.

Prior patents relating to the use of microarrays for nanofabrication include the following, all of which are hereby incorporated in by reference as if fully set forth herein: U.S. Pat. No. 6,652,808 entitled “Methods for the Electronic Assembly and Fabrication of Devices”, U.S. Pat. No. 6,569,382 entitled “Method for the Electronic, Homogenous Assembly and Fabrication of Devices”, and U.S. Pat. No. 6,706,473 entitled “Systems and Devices For Photoelectrophoretic Transport and Hybridization of Oligonuceotides”.

### SUMMARY OF THE INVENTION

A new, extremely sensitive, and rapid electronic detection method for direct detection of hybridized genomic targets to specific probes on the microarray is proposed. The method consists of fast electronic accumulation of the DNA target on a particular electrode site at the micro-electrode array, sequential electronic hybridization of oligonucleotide labeled metallic (nano)particles on the target DNA and monitoring the electrochemical AC impedance changes at the electrode site. The method is enhanced by electroplating over the DNA target which serves as the metallization template and over the particles which provide seeds for rapid electroplating. The AC impedance changes are monitored during the electroplating over the DNA target and between the array electrodes sites. The signal in the absence and presence of the target DNA is a difference between “no connection” and a “short” between the array electrodes. This assures an extraordinary signal-to-noise ratio. The method offers unprecedented sensitivity, theoretically approaching single or only a few DNA molecules attached to the electrode site. Rapid electronic addressing of the DNA target and labeled nanoparticles to the microarray assures that the detection at these levels of sensitivity will be achieved within only a few minutes.

Applications of the innovations used in the electronic detection system include at least the following: electronic capturing of target DNA on the electroactive microarray and electronic alignment of labeled particles as seeds for DNA-templated electroplating, sequential or cyclic electrophoretic accumulation of labeled particles on DNA target as tags for AC impedance signal amplification—cyclic electrophoretic AC signal amplification, and DNA-templated electroplating on the electroactive microarray—electroplating of target DNA between the electroactive array sites over the labeled metallic particles and/or directly in the presence of electroplating ions, e.g., Ag, Au, Pd.

Portable DNA analysis systems for molecular diagnostics is the integration of the sample preparation and detection steps on a single platform. This invention includes an electronic detection technique for the microarray technology which will be capable of easy integration with various sample preparation methods including those based on magnetic particles.

The target DNA-templated electroplating detection system which utilizes electrochemical impedance spectroscopy between the electrode array sites as the microarray detection signal presents an innovative approach to DNA sensing. However, the detection technique builds on similar, established, and demonstrated electro-less techniques for DNA metallization which utilize charge interactions between the metallic ions and DNA and subsequent reduction of attached metal ions. Other such techniques utilize micro- or nanoparticles attachment to the DNA structure to achieve a layer of metallic particles which are then passively coated using a different set of metallic particles. These techniques often take hours to implement the DNA plating process and are not site specific. The unsurpassable advantage of the proposed detection system is that the DNA target as well as the metallic particle tags are very rapidly and specifically addressed at the electroactive microarray, they can be easily accumulated at a particular array site and AC signal enhanced in a cyclic electrophoretic accumulation of particle tags. This unique and rapid signal enhancement by electrical alignment and electronic formation of metallic particle clusters on the target DNA assures an easily measurable electrochemical impedance changes on the electrode site. In addition, the electronically aligned particles enable fast seeding of the DNA template as well as extremely accurate DNA electroplating. The use of direct and sequence specific electroplating of DNA, instead of slow electro-less plating techniques, is proposed here for the first time.

The electroactive transport allows attachment of metallic tags on DNA in a cyclic and amplifiable manner where each cycle occurs within only a few seconds. The basic electronic microarray technology will allow development and unrestricted practicing of this new “electrophoretic amplification” technique for rapid enhancement of the DNA signal. A similar amplification technique may be used in a fluorescence based detection where the DNA and fluorophore attached tags are accumulated and amplified using electroactive transport. This project will focus on AC impedance based detection of the signal that is highly compatible with our electronic microarray technology.

### BRIEF DESCRIPTION OF THE DRAWINGS

FIG. 1 shows a schematic diagram of the proposed electronic detection system for specific and highly sensitive detection of DNA targets.

FIG. 2 shows how the AC signal monitoring is performed on the microarray.

FIG. 3 shows the use of several types of metallic particle tags.

FIG. 4 shows simultaneous detection of all five HPV types.

FIG. 5 demonstrates that it is possible to perform simultaneous on-chip SDA amplification of up to 10 different genes in a single sample.

FIGS. 6a and 6b show the AC impedance spectra which demonstrates changes in capacitive and resistive components occurring between two electrode array sites (the locations 1,1 and 1,10 are shown; the first number designates row and the second number designates colurn in the microarray) at two working electrode potentials applied with respect to the chip reference electrode and as a function of the histidine supporting electrolyte concentration.

FIG. 7. Nanogen's portable prototype instrument with the electroactive micro-array and optical detection. The instrument is operated by a laptop (left). Components of the instrument include the cartridge inlet port, reagent reservoirs, peristaltic pumps, electronic control and optical detection system with a CCD camera (right).

FIG. 8. Photograph of the 400-site CMOS ACV400-chip cartridge and array. Four counter-electrodes, two longitudinally and two horizontally positioned surround the active working electrode array.

### DETAILED DESCRIPTION OF THE INVENTION

AC Impedance System for Detection of DNA-Templated Electroplating

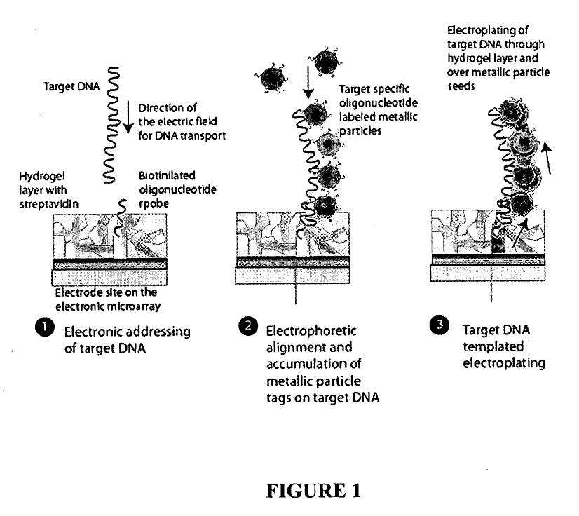

FIG. 1 shows a schematic diagram of the proposed electronic detection system for specific and highly sensitive detection of DNA targets. The detection consists of the following steps.

Electronic addressing of the target DNA occurs first. This step occurs in accordance to Nanogen's developed technology and implies accumulation of low concentration of DNA targets at an electrode array site from solution by electrophoresis. The electronic microarray is covered by a hydrogel permeation layer (ca 7-10 micron thick) containing streptavidin molecules. The proposed system assumes the use of pre-loaded biotinilated probes complementary to a particular gene region of interest on the target DNA. The target DNA can be very rapidly, within less than one minute, accumulated from the solution and electronically hybridized at a particular array site, providing a localization of the detection process.

Once the target DNA molecules are hybridized to the oligonucleotide probes at the electrode, the oligonucleotide labeled metallic particles are hybridized along the captured target DNA. The sequences of the oligo probes on the particles will vary depending on the size of the DNA template and the diameter of particles, thus providing a proper spacing between the particles on the DNA for subsequent metallization. The use of oligo-labeled particles offers an additional level of specificity thus reducing the non-specific binding and occurrence of eventual false positives to a minimum.

The non-captured particles are washed away and those captured on the DNA target(s) are electrophoretically aligned, thus providing a series of seeding sites for continuous metallization.

Nanogen's technology allows precise spatial capturing of DNA targets at a particular microarray site and this feature is used further in the proposed system for localized DNA-templated electroplating. The electroplating occurs first through the nanopores (diameter ca 50-200 nm) of the thin permeation layer and proceeds to the first and then subsequent metallic particles aligned (hybridized) along the DNA target. Because the size and number of particles can be optimized with respect to the DNA target, the electroplating process can be accomplished within only few minutes.

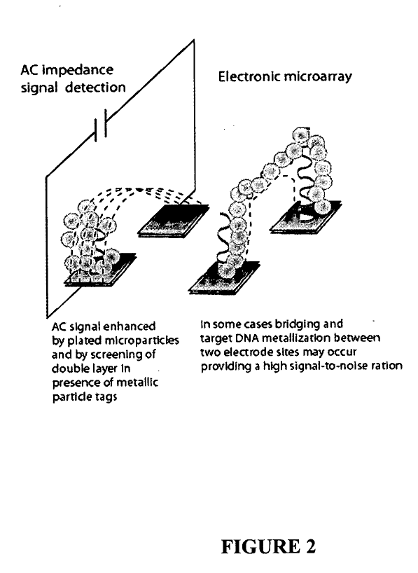

The accumulation of metallic particles at the captured DNA template can be followed through changes in the AC impedance signals at the electrode site. FIG. 2 shows how the AC signal monitoring is performed on the microarray. The electrochemical double layer is formed on the electrode where the DNA target is accumulated and extends through the pores of the hydrogel permeation layer. As the metallic microparticles electrophoretically accumulate on the DNA target template they screen the electric field lines extending through the solution between the two electrode array sites and particularly change the capacitive and/or resistive components of the impedance of the working electrode (the electrode where the DNA target is addressed). Each metallic particle possesses its own electrochemical double layer. The thickness of the electrochemical double layer (EDL) typically ranges from 10 nm to 100 nm (See Reference(s) 49). Thus each particle can further disturb the impedance signal of the electrode through its own capacitive component of the particle EDL. In addition, the aligned metallic particles can act as a series of bi-polar electrodes inserted between the two electrode array sites.

These processes could accelerate the electroplating process over the seed-particles as well as affect the electrode EDL. It is envisioned that the AC impedance signal will change significantly as the particles accumulate even without the electroplating process. However, for higher signal sensitivity, the aligned metallic particles are electroplated. They become electronically connected to the electrode array site and effectively extend the working electrode surface area. The use of nanoparticles will significantly change the electrochemical surface area of the working electrode and provide an easily measurable AC impedance signal change. The proposed detection technique envisions even further signal amplification which occurs when the metallic particles bridge the gap between the two electrode array sites. The bridging can be promoted by using relatively long oligonucleotide probes at the electrodes as well as by minimizing the electrode spacing.

FIG. 2 shows an AC impedance monitoring of the target DNA-templated electroplating process using electrophoretically accumulated and aligned metallic particles along the DNA target captured at a particular array site. Two cases are shown, one that demonstrates the changes in the electrode impedance due to clustering of metallic particle tags and their effect on the electric field lines (dashed lines) (left) and the other when the metallic particles bridge the gap between the electrode sites (right).

Poly-T Embodiment

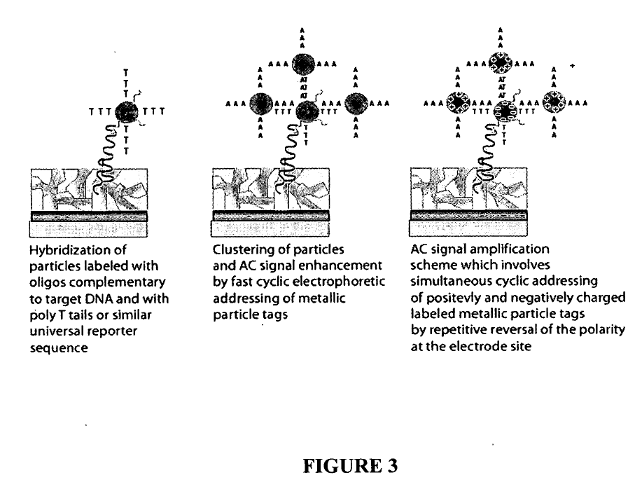

There is yet another signal amplification technique that is a part of the proposed electronic detection technique and can be used if very low concentration of DNA target needs to be detected. FIG. 3 shows the use of several types of metallic particle tags. The first step includes electrophoretic addressing of metallic particle labeled with both oligonucleotides having a complementary sequence to the target DNA and oligonucleotides having a simple repetitive sequence such as poly-T tails. Other simple sequences could be used. A second type of metallic particle tags contains oligonucleotides complementary to the poly T, i.e., a poly A sequence (or similar simple sequence complementary to the sequence on the first set of particle tags). The method implies a repetitive electrophoretic addressing of metallic particle tags which in subsequent addressing steps hybridize between themselves, thus promoting a fast clustering of metallic particles at the electrode site where the DNA target is captured. This will cause dramatic changes in the AC impedance signal because a large percentage of the electrode area could be covered quickly. This new “electrophoretic amplification” of the signal uses fast electrophoretic addressing of multiple particle tags in several separate steps or cycles (a washing step may be needed between the additions of particle tags). Because the second addition or the second cycle already provides a chain-like hybridization between the particle tags, it is envisioned that only few such cycles may be needed to obtain a high signal-to-noise ratio. The electrophoretic addressing in each cycle will take only a few tens of seconds, thus the entire cyclic amplification process will be no longer than 3-5 minutes. This new signal amplification technique can yield to an extremely fast and highly sensitive DNA detection system.

The cyclic electrophoretic addressing also implies the addressing of particle tags of opposite charge. Some metallic particle tags can be made negatively charged (e.g., carboxylated particles) or positively charged (e.g., aminated particles). These particle tags will contain the same type of oligonucleotide labels as described above. The advantage of this approach is that once the DNA template is electronically hybridized and anchored to the permeation layer, these metallic particles can be addressed in a faster, electrochemical “stirring” mode by repetitively reversing the polarity of the two electrodes (one contains the DNA target the other is the counter electrode). The tags in the second or third cycle could be added simultaneously and the chain-like hybridization and clustering induced by a polarity reversal.

FIG. 3 shows enhancement of the AC impedance signal through cyclic electrophoretic hybridization of various metallic particle tags capable of a chain-like hybridization between themselves. This can occur in only a few fast cycles as well as by using the particles of an opposite charge and by reversing the polarity of the electric field applied at the electrode site.

Experimental

Nanogen, Inc. has previously designed and developed miniaturized and integrated systems for microarray-based DNA detection (See Reference(s) 50-52). Nanogen's technology for DNA detection (commercial Nanochipg electronic microarray system) enables rapid and accurate determinations of single nucleotide polymorphic mutations (See Reference(s) 53). Nanogen offers commercial analyte specific reagents for the diagnosis of a number of coronary and hemochromatosis diseases (e.g., Factor II, Factor V, Factor V/II combination assay, cystic fibrosis, HFE, Canavan disease and ApoE gene—late onset of Alzheimer's disease). Nanogen's platform is a unique and open platform which allows customers to create their own arrays and assays. Customer list of applications based on SNP determination using our platform includes: coronary artery diseases, cardiovascular disease, hypertension, cardiac function, cancer applications, bacteria identification, multidrug resistance, hemophilia, Thalassemia, etc.

Sensitive Detection of Infectious Disease Pathogens Using Electronic Microarray

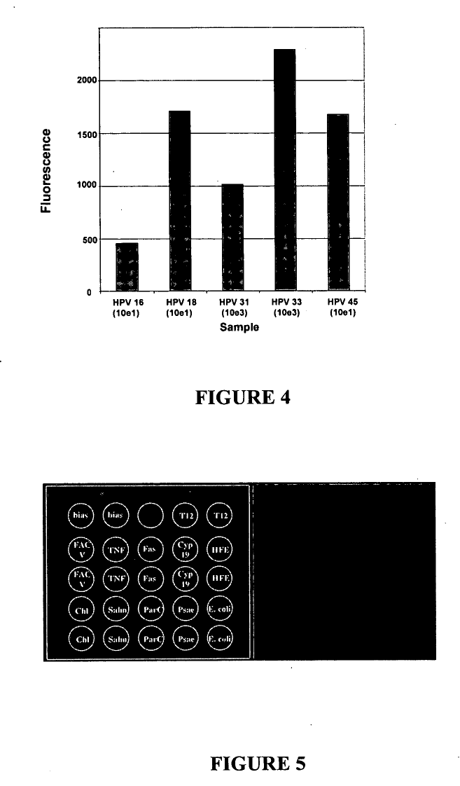

This section summarizes recent studies performed at Nanogen to demonstrate efficient electronic accumulation of PCR and SDA (strand displacement) amplified DNA targets on the electronic microarray and its detection using current fluorescence based detection. A feasibility study was performed using five Human Papillomavirus, HPV types (HPV 16, HPV 18, HPV 31, HPV 33 and HPV 45). The amplification was performed using PCR (AmpliTaq® Gold) in 25 μL reactions on a Perkin-Elmer 9700. Detection was performed on a 100-site chip. FIG. 4 shows simultaneous detection of all five HPV types. All five types clearly show significant signal above the background signal. Three out of five of the HPV types were present at only 10 copies).

FIG. 4 shows fluorescence data obtained on 100-site electronic microarray for detection of five HPV types amplified using PCR. Detection as low as 10 copies of each HPV type was demonstrated.

A multiplexed PCR-based assay for Bacillus anthracis and vaccinia was developed and an independent validation was performed by our collaborator, Midwest Research Institute. Testing included evaluation of screening assays and confirmation assays using hemagglutinin gene for vaccinia and CapB and protective PA genes for anthrax. Specificity of the assays was evaluated against a panel of 28 anthrax strains and near neighbors of B. anthracis, vaccinia, rabbitpox, raccoonpox, and a number of other select agents including Francisella tularensis, Yersinia pestis, Clostridium botulinum, and Erwinia Herbicola. The procedures included: (i) overnight growth of B. anthracis strains (available from ATCC), vaccinia, and all competitive strains used; (ii) extraction of their DNA using bead beating, centrifuging and elution in accordance to commercial kits (modified Qiagen kits); (iii) DNA quantitation (PicoGreen dsDNA Quantitation kit, Molecular Probes), and (iv) performance of: a) screening assay; b) confirmation, competition assays, and c) specificity assays. The experiments were conducted under BSL 3 safety conditions when needed. The limits of detection (LOD) were determined for the range between 0.17 to 1,700 copies of B. anthracis strains (per PCR reaction) or 0.0015 to 1,500 PFUs for vaccinia using serial dilutions of quantified DNA. (50 microliter PCR reactions were performed on a PE 9600 thermocycler and detection accomplished on Nanogen's 100-site electronic microarray). Testing of B. anthracis (Vollum strain) demonstrated a limit of detection of 1 pg or 170 copies for the CapB screen assay (100% positive results for 20 replicates), and 10 pg or 1,700 copies for the PA gene. The confirmatory assay for the CapB gene showed LOD of 100 fg or 17 copies (100% positive results for 20 replicates). Testing of vaccinia, ATCC VR-2010, with the Hema assay demonstrated an LOD of 15 PFU (plaque forming units) The specificity testing (see list of near neighbors and other select agents tested above) demonstrated that positive results were obtained only when target genes CapB, PA, or Hema were present in the sample and no other agents inhibited the positive results. One ng DNA per reaction (170,000 copies) was used in the specificity testing.

On-chip Strand Displacement Amplification—Demonstration of a Highly Efficient Accumulation of DNA Targets

We have developed a number of assays using an isothermal Strand Displacement Amplification (See Reference(s) 54-55). (SDA, licensed from Bectkon Dickinson) of DNA targets because this method requires a much simpler device for thermal control in a portable instrument compared to thermal cyclers used for the PCR amplification. In the SDA amplification DNA polymerase recognizes the nicked strand of DNA and initiates re-synthesis of that strand, displacing the original strand. The released amplicons then travel in solution to primers for the complementary strand which are either in solution or anchored. Oligonucleotide primers without nicking sites called bumper primers are synthesized in the regions flanking the amplicons just produced, and assist in strand displacement and initial template replication. A typical reaction mix for SDA amplification consists of the following materials.: a) sense and antisense primers 500 nM; b) Bumper primers 50 nM; c) dNTP mix 1.4 mM each; d) Bst polymerase 9.6 U/rxn; e) Bbv nicking enzyme 3.75 U/rxnMg(OAc); f) 10 mM pH 7.6 phosphate buffer, 25 mM. Generally, the reaction volume is 10-50 μl. These parameters are optimized through the Design of Experiment (DOE) optimization of experimental parameters. We have developed anchored SDA amplification method where the internal amplification primers (not the bumper primers) are biotinylated and addressed to specific electronic microarray sites where they bind to the streptavidin in the hydrogel permeation layer. These primers can be pre-loaded on the chip at a manufacturing stage. Preliminary stability experiments performed in the period of ca 2 months demonstrated good stability of pre-loaded primers. This step will be important in accelerating the assays and performing the addressing of targets and reporters only. The target DNA is then addressed to the array site where it electronically hybridizes to the anchored primers. Finally, the microarray is covered with a reaction mixture containing enzymes, bumper primers and dNTP's and heated to 50° C. for 30 minutes to an hour to obtain the reaction products.

FIG. 5 shows a 10-plex on-chip SDA amplification. The pattern of amplified genes is shown on the left. On the right is a fluorescence image of the microarray after amplification and reporting. (Nature Biotechnology, Feb. 2000).

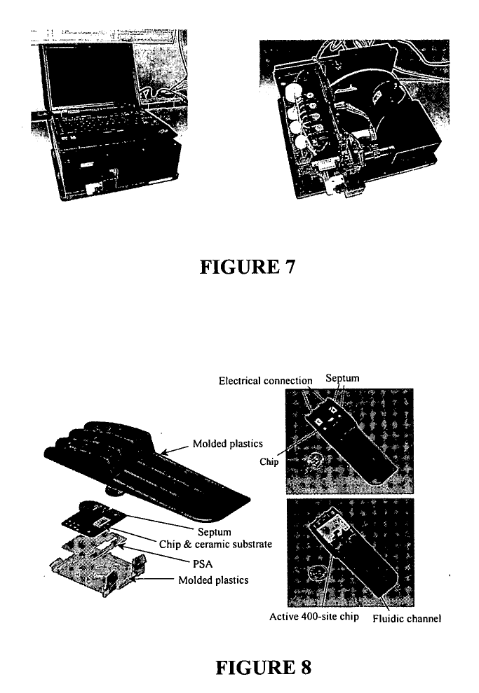

FIG. 5 demonstrates that it is possible to perform simultaneous on-chip SDA amplification of up to 10 different genes in a single sample. The experiment shows multiplexing of 5 human and 5 bacterial genes relevant to identification of infectious diseases and/or biological warfare agent on the electronic microarray. A number on-chip SDA based assays were developed for the detection of infectious pathogens and/or biological warfare agents using our miniaturized prototype microarray detection instrument (shown in FIG. 7). The following 6 genes for four biological warfare agents were analyzed: bacillus anthracis (anthrax) (cap B and PA genes), vaccinia (hemagglutinin gene), Staphylococcus aureus (sea and seb genes) and plague (Yersinia pestis) (plasminogen activator, PLA gene). A range of concentrations of each DNA was addressed before the anchored amplification. Concentrations as low as 85 copies of DNA/microliter (in the detection chamber) could be commonly detected for vaccinia. For the B-list CDC agents, e.g., E. Coli and S. typhymurium, results obtained reproducible anchored SDA data in the range between 10-100 copies of DNA (with respect to the starting volume of the amplification reaction). Very recently, we have demonstrated that as low as 5 copies of the vaccinia DNA target gene per array electrode can be efficiently accumulated within 1 minute of electronic addressing time (results obtained using a portable instrument). Positive results were obtained on 18 addressed electrodes using 85 copies/microliter on the chip (yielding 85/15 ca 5 copies per electrode). The result was obtained after SDA amplification of the DNA concentration on each electrode array site. This demonstrated that the electronic addressing is efficient enough to be used in the proposed direct amplification-less detection technique where only a few DNA molecules present in the sample can be efficiently captured on the electrode array site. This resolves one of the problems in the proposed detection technique, i.e., a demonstration that one or few DNA target molecules can be attached to the array site within a very short period of time (one minute).

AC Impedance Measurements on the Electronic Microarray

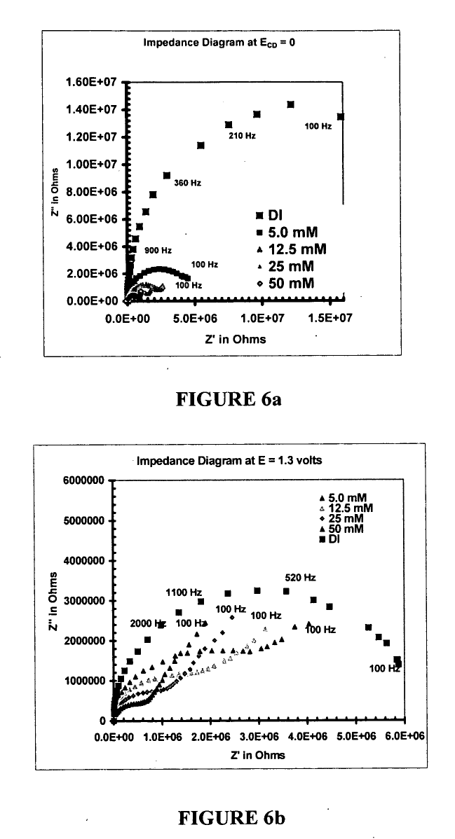

We have performed initial AC impedance measurements between the electrode array sites on the 400-site microarray in conditions where electrophoretic DNA accumulation is promoted. The AC impedance spectra shown in FIG. 6a and 6b demonstrate changes in capacitive and resistive components occurring between two electrode array sites (the locations 1,1 and 1,10 are shown; the first number designates row and the second number designates column in the microarray) at two working electrode potentials applied with respect to the chip reference electrode and as a function of the histidine supporting electrolyte concentration. The spectra exhibit typical Randles equivalent circuit circular shape (cf., FIG. 6). By increasing the concentration of histidine the semi-circles become smaller, indicating a higher current due to higher concentration of the electroactive species in solution. Polarization resistance, Rp, and solution resistance, R_{s,} were calculated for all impedance spectra using a least-square method fit for a semicircle as shown in FIG. 6. At low conductivity, i.e., at low electrolyte concentrations, the Rₚ/Rₛ ratio at E_{DC}=0.0 V is up to two orders of magnitude higher compared to higher electrolyte concentrations. This trend is also observable at E_{DC}=1.3 V, although the decrease in the Rₚ/Rₛ ratio with concentration is smaller, i.e., up to 10-fold decrease. The results clearly demonstrate that at low electrolyte concentrations the total resistance is very high, and is dominated by the polarization resistance. Consequently, the total currents at the electrode array are very small and solution impedance can be affected by the geometrical arrangement of the electrodes. These impedance characteristics precisely describe relevant conditions of our assays on the electronic microarray. The data indicate that the electrode/electrolyte interface on the array sites will be significantly affected by the presence of adsorbing species on the electrode as well as by any changes in the electrode geometry. Accumulation of metallic nanoparticle tags, in particular their exponential amplification in the chain-like electroplating of the DNA target, can dramatically increase the electrode electrochemical surface area and yield an easily measurable impedance signal dependent on the DNA concentration accumulated at the array site.

Research Design and Methods

The overall objective of the proposed research is to demonstrate the feasibility aspects of the development of a new microarray detection platform that uses electronic addressing of a low copy number of DNA targets and its detection using the electrochemical AC impedance signals of the metallization process of the DNA target molecules attached to the array sites. Metallic particle tags are used to enhance the AC signal during the DNA-templated electroplating and the signal is amplified through a cyclic electrophoretic addressing of the particle tags. The electronic detection system enables the cartridge as well as the instrument packaging in a miniaturized format which will allow development of a portable instrument. The proposed research will leverage our previous efforts in the development of portable DNA microarray instrumentation and an existing prototype miniaturized platform containing the necessary fluidics; electronic and software components will be adapted and used in the validation of the proposed detection system. The following are the specific technical objectives of the proposed research.

AC impedance detection of the DNA metallization process in the presence of metallic particle tags.

Design and fabrication of an electronic microarray and cartridge with the electrode array geometry suitable for the proposed detection system.

Demonstration of amplification-less, rapid and sensitive detection of DNA target molecules.

Design and testing of a representative assay and validation of the detection system.

Experiments planned in the proposed research and development effort will be entirely performed at Nanogen's facilities. The masks for the fabrication of microarray chips will be outsourced to a silicon micromachining foundry and the fabrication of the array and cartridge will be made in-house using methods and vendors established for our commercial equipment. Nanogen has all the necessary equipment, microfabrication facilities (clean rooms, class 100 and 1000), microbiology and molecular biology labs as well as personnel available to perform all the tasks of the project.

AC Impedance Detection of the DNA Metallization Process in the Presence of Metallic Particle Tags

Electrochemical impedance spectroscopy (See Reference(s) 56) utilizes a small 10-50 mV sinusoidal potential signal applied in a range of frequencies (from few micro-Hertz to MHz range) at the working electrode to determine the capacitive and resistive components at the electrode/electrolyte interface. The method allows a mechanistic insight into the structure of the electrochemical double layer (capacitive behavior), discriminates faradaic or electoractive components of the current and difflusion controlled processes as well as it provides resistive or capacitive behavior of a coating or adsorption on the electrodes. The impedance is usually expressed as a complex ftnction (cf., Eq 1-3) and data are represented using Nyquist plots where real or resistive components are presented on the X-axes and imaginary or capacitive components are represented on the Y-axes (cf., FIG. 6). Bode plots are used to examine a phase shift and absolute value of impedance as a function of frequency. An electronic equivalent circuit is usually established which provides a model of the interface and helps with understanding the dominant real time (resistive) or imaginary (capacitive) components of the impedance signal as the experimental conditions are varied.
E(t)−E₀ exp(jωt) (1)
I(t)−I₀ exp(jωt−jφ) (2)

Where E is a sinusoidal potential applied, and I is the current response, ω is angular velocity. The impedance can then be represented as the complex number:
Z=E/I=−Z₀ exp(j, φ)=Z₀(cos φ+j sin φ) (3)

AC impedance was recently used to detect an antibody/antigen binding effect at the flnctionalized electrodes (See Reference(s) 57). These results showed 25% difference in AC signal comparing electrodes flnctionalized with a specific antibody and another electrode with a control (non-binding) antibody. These examples as well as some of our preliminary data, demonstrate that the AC impedance studies are suitable to monitor the adsorption or deposition processes occurring directly on the surface of the electrodes, thus reflecting minimal changes in the surface area of the working electrode. The particle accumulation, especially when enhanced by cyclic electrophoretic addressing on DNA target molecules, will affect the electrochemical double layer extending through the hydrogel layer nanopores and cause substantial impedance changes during electroplating of DNA targets and metallic particle seeds.

The following is a rationale of the proposed experiments: 1. AC impedance spectra will be established and compared in the presence and absence of captured DNA targets on a number of microarray electrode sites and reproducibility of the signal established; 2. Fundamental changes of impedance parameters will be investigated for the electroplating process at a particular electrode site; 3. Impedance signals will be determined for DNA-templated electroplating process in the presence and absence of metallic particles addressed at the DNA target; 4. Dependence of the most prominent impedance component of the AC signal will be examined as a function of the concentration of DNA added to the array fluidic chamber. The DNA targets will include several levels of complexity: a) initial optimizations will be performed with PCR amplified and purified genomic DNA (size in the range 200-1,000 bp) with known sequences (genomic DNA available from ATCC; DNA targets and designed primers are available for a number of pathogens, e.g., Yersinia pestis, pla gene, Lysteria monocytogenes, hly gene, Streptoccocus pneumoniae, ply gene, anthrax, several genes, etc.); b) once the conditions are optimized, genomic DNA (5-6 Mbp) will be tested targeting characteristic gene sequences; c) a complete assay will be tested using genomic DNA in Task 4.

Fundamental impedance studies will be performed using an Autolab Eco Chemie potentiostat/frequency response analyzer, Model PGSTAT20 (several are available at Nanogen). The impedance parameters will be examined in the following range: sinusoidal AC signal at 10-50 mV amplitude; frequency range 20 kHz to 5 Hz between the array electrodes. DC potential (E_{DC}) will be controlled with respect to Ag/AgO QRE surrounding the electrode array and/or versus an external standard calomel electrode. The buffer electrolytes will include our standard buffers for the performance of electronic microarray DNA analysis including histidine (concentration 10-100 mM), low salt-buffer (phosphate buffer), and a high salt buffer (phosphate buffer with sodium chloride ions). We have developed fixtures which can provide contacts to the cartridge and the chip. The 400-site miniature prototype system developed as a part of the DUST program (cf., FIG. 7) which accepts a 400-site array/cartridge (cf., FIG. 8) will be used to perform electronic addressing as well as further impedance measurements. The system has a built in fluorescence detection system which will be used as a verification of the hybridization of particles to the DNA target (fluorescence labeled particles will be used for this purpose) as well as a DNA attachment to the oligonucleotide probes in the permeation layer. The CMOS chip has an array of 16×25 (400-sites); each electrode being 50 μm in diameter with a 150-μm center-to-center distance. The CMOS chip is a flip-chip bonded onto a ceramic substrate (0.015″ thick), which is further bonded with a machined cover plate (acrylic) by pressure sensitive adhesive into a cartridge (cf., FIG. 8). Assuming the length of DNA at ca 0.34 nm per base pair, DNA bridging experiments will use 20-40,000 bp DNA templates. The size of these DNAs is therefore in the range between 2-4 microns and they will be capable of bridging the electrode distance of the newly designed chip.

FIG. 7. Nanogen's portable prototype instrument with the electroactive micro-array and optical detection. The instrument is operated by a laptop (left). Components of the instrument include the cartridge inlet port, reagent reservoirs, peristaltic pumps, electronic control and optical detection system with a ccd camera (right).

FIG. 8. Photograph of the 400-site CMOS ACV400-chip cartridge and array. Four counter-electrodes, two longitudinally and two horizontally positioned surround the active working electrode array.

Design and Fabrication of an Electronic Microarray and Cartridge with the Electrode Array Geometry Suitable for the Proposed Detection System

The main goal of this task is to develop a microelectronic array with the electrode geometry that will assure highest AC signals for the proposed detection mechanism. This may involve decreasing the spacing and diameter of the electrodes. It is envisioned that the diameter of the electrodes in the range between 3-5 microns with approximately similar spacing will provide a higher AC signal, in particular it will increase chances to achieve the bridging between the two electrode sites. This level of line resolution can be achieved using the same lithographic techniques used in the production of the current 400-site array (RF sputtering for platinum deposition and plasma enhanced chemical vapor deposition techniques (PECVD) for insulating silicon dioxide deposition). All the equipment is available and methods established at Nanogen for a production of such chip. The mask fabrication and flip-chip processes will be performed using standard vendors. The hydrogel permeation layer is fabricated in-house using automated micro-molding and UV curing equipment. The experiments in this task will involve the use of larger oligonucleotide probes for bridging the gap (e.g., 1,000-10,000 based pairs) between the neighboring electrode array sites as well as smaller nucleotides probes hybridized to those probes having sequences specific to the template DNA.

Demonstration of Amplification-Less, Rapid and Sensitive Detection of DNA Target Molecules

This task will focus on defining and testing other important parameters for the optimization of the AC impedance characterization of the DNA target metallization. The DNA targets will be relatively short oligonucleotides mimicking the PCR amplified DNA samples. Their length will range between few tens to few hundred base pairs. The shorter templates will be obtained using PCR amplification and purification of the product. We have a number of DNA templates which are used as well controlled DNA samples such as Factor II or Factor V sequences. The oligonucleotide probes will range between 50-80 base pairs to assure a high specificity for the DNA template. The experiments planned in this tasks will involve optimization of the metallic particles tags with respect to: a) particle diameter—ranging from 10 nm to 1 μm diameter; b) charge of particles: carboxylated particles with positive and aminated particles with the negative charge will be used in the cyclic electrophoretic measurements with electrode polarity reversal to enhance the AC signal as explained earlier; c) oligonucleotide labels: one set of particle tags will be labeled with both sequences complementary to the DNA target as well as with poly-T (or similar repetitive sequence) for reporting and hybridization to other particle tags in subsequent electrophoretic addressing (amplification) cycles; other particle tags (subsequent in addition to the first set) will contain poly-A oligo labels with the sequence complementary to poly-T (or similar complementary sequence); d) concentration of oligonucleotide probes coverage on the particle tags—it is envisioned that there will be an optimum of oligo probes concentration on the particles with respect to achieving an efficient clustering of particle tags on the DNA template; e) the number of particle tags with oligonucleotide probes complementary to the DNA target template—the number of complementary tags aligned along the DNA will be optimized with respect to the size of DNA target and size of particles to achieve conditions for fastest seeding and most efficient metallization.

Several binding techniques between the particle and oligo probes may be tested; however, commercially available particles will be used whenever possible. DNA oligonucleotide probes can be covalently coupled directly to the beads to get a surface coverage ranging from only few probes to as large as 10⁵ per particle. The oligonucleotide particle tags could consist of poly dT, poly dA, poly dC or poly dG sequences. In addition, specific capture sequences can be added to these beads or a mixture of beads may be used. Alternately the beads can be covalently modified with streptavidin and then used to bind biotinylated oligonucleotide probes. Carboxyl and amine terminated particles, and amine terminated quantum dots are available from several vendors including Polysciences, MoSci, Nanosphere, Pierce, Seradyn, Dynal and Quantum Dot. Several reagents can be used to covalently couple streptavidin or DNA directly to the beads. For positively charged, aminated beads glutaraldehyde can be used to activate the beads followed by the addition of 5′ or 3′-amino modified DNA sequences at the right concentration to achieve a controlled density of probes per bead. Alternatively, streptavidin can be added to the glutaraldehyde activated beads and covalently coupled through the terminal lysine residues on the streptavidin subunits. The linked beads are then passivated using monoethylamine to maintain a net positive surface charge and to react with the remaining aldehyde linkages.

For negatively charged, carboxylated, beads 1-ethyl-3-(3-dimethylaminopropyl) carbodiimide (EDC) can be used to activate the beads followed by the addition of 5′ or 3′-amino modified DNA sequences at the appropriate dilution to achieve a controlled density of probes per bead. Alternatively streptavidin can be added to the EDC activated beads and covalently coupled through the terminal lysine residues on the streptavidin subunits. The linked beads are then passivated using glycine to maintain a net negative surface charge and to react with the remaining o-acylisourea linkages.

Large size DNA templates will be used to test the probability and conditions in DNA template/oligonucleotide probe bridging experiments. Those may include cloned plasmids in the size range from 5 to 20 kb base pairs available commercially (e.g., Invitrogen offers lyophilized plasmids in variety of sizes, e.g., pREP4, an episomal mammalian expression vector, Catalog #V004-50, has 10.3 kb. A series of restriction enzymes are provided which can be used to cut the plasmid to a desired length (e.g., Aatl will provide only one cut on pREP4). The plasmids with known sequences will be used which will simplify the design of the oligonucleotide probes for sensor applications. These longer probes will be attached to neighboring electrodes to provide longer arms for bridging with the DNA template and extended particles tags. Once metallized (as described earlier), the current will flow over the metallized bridge and provide an extremely high impedance signal-to-noise ratio because a short will be created between the two electrodes. It is noteworthy that the bridging between the two electrode sites could be made of several pieces of single stranded DNA attached to each other at their ends or through metallic particle tags, thus providing a longer stretch between the electrodes.

Design and Testing of a Representative Assay and Validation of the Detection System

To properly validate the proposed detection method DNA target samples with accurately known sequence will be used in the assay design. We have several relevant plasmid constructs as well as genomic DNAs with known sequences, e.g., vaccinia plasmid, or pla plasmid (plague) which were obtained from USAMIID. This task will examine aspects of performing an entire assay including the potential for integration with the sample preparation steps. The portable instrument developed through the DUST program could accommodate both sample preparation and proposed new detection system.

It is envisioned that the antibody or oligonucleotide labeled magnetic particles could be used in the proposed detection technique. This will enable integration with simple sample preparation steps which will consist of magnetic separation of pathogens from the sample using antibody labeled beads (through the DUST program we have developed a number of antibody labeled beads specific for several infectious disease pathogens, e.g. E. Coli, S. typhimurium, S. pneumoniae, etc). The pathogens (or cells of interest) are thus first separated magnetically and subjected to lysis (we have demonstrated that simple thermal lysis steps were satisfactory to efficiently separate and confirm pathogen levels as low as 10-100 per ml). If necessary, released genomic DNA could be first enzymatically cut to a precise number of cuts with known sequences. The DNA target is captured on the microarray by electronic addressing to the biotinilated oligonucleotide probes on the hydrogel permeation layer. The oligonucleotides labeled metallic particles are then added and the assay performed as described earlier for the AC impedance detection of DNA target metallization. This task will result in the optimization of the assay steps and will evaluate ruggedness and reproducibility as well as the sensitivity of the proposed electronic detection method. The validation performed for the PCR amplified sequences of interest, DNA size 200-1,000 bp as well as for the representative genomic DNA (4-6 Mbp, cut in pieces enzymatically or by thermal treatment in the sample preparation process).

It will be apparent to those skilled in the art that modifications may be made without departing from the spirit and scope of the invention. Accordingly, it is not intended that the invention be limited except as may be necessary in view of the appended claims.

References Cited
1. U.S. Pat. No. 5,532,129 “Self-Organizing Molecular Photonic Structures Based on Chromophore and Fluorophore Containing Polynucleotides and Methods of Their Use”, Jul. 02, 1996
2. M. J. Heller, IEEE Engineering In Medicine and Biology, March/April, 100 (1996).
3. U.S. Pat. No. 5,565,322 “Hybridization of Polynucleotides Conjugated with Chromophores and Fluorophores to Generate Donor-to-Donor Energy Transfer System”, Oct. 15, 1996
4. U.S. Pat. No. 5,605,662 “Active Programmable Electronic Devices for Molecular Biological Analysis and Diagnostics”, Feb. 25, 1997
5. U.S. Pat. No. 5,835,404 “DNA Optical Storage Device Utilizing Non-Radiative Energy Transfer”, Nov. 10, 1998
6. U.S. Pat. No. 6,652,808 “Methods for the Electronic Assembly and Fabrication of Devices”, Nov. 25, 2003
7. U.S. Pat. No. 6,706,473 “Systems and Devices for Photoelectronic Transport and Hybridization of Oligonucleotides., Mar. 16, 2004”
8. U.S. Pat. No. 6,726,880 “Advanced Active Electronic Devices for Molecular Biological Analysis and Diagnostics and Methods of Manufacturing Same”, Apr. 27, 2004.
9. Kricka, L. J. Revolution on a square centimeter, Nature Biotechnology, 16: 513-514, 1998
10. Electric field controlled preparation and hybridization analysis of DNA/RNA from E. coli on microfabricated bioelectronic chips, Cheng, J., Sheldon, E. L., Wu, L., Uribe, A., Gerrue, L. O., Carrino, J., Heller, M., and O'Connell, J.: Electric field controlled preparation and hybridization analysis of DNA/RNA from E. coli on microfabricated bioelectronic chips. Nature Biotechnology, 16: 541-546, 1998
11. S. K. Kassegne, H. Reese, D. Hodko, J. M. Yang, K. Sarkar, D. Smolko, P. Swanson, D. E. Raymond, M. J. Heller and M. J. Madou, Sensors and Actuators B, 94, 81 (2003).
12. Y. Huang, E. L. Mather, J. L Bell, and M. Madou, Anal. Bioanal. Chem. 372, 49 (2002).
13. L. Westin, X. Xu, C. Miller, L. Wang, C. F. Edman, and M. Nerenberg, Nature Biotechnology, 18, 199 (2000).
14. J. M. Yang, J. Bell, Y. Huang, M. Tirado, T. Thomas, A. H. Forster, R. W. Haigis, P. D. Swanson, R. B. Wallace, B. Martinsons, and M. Krihak, Biosen. Bioelect. 17, 605 (2002).
15. S. S. Iqbal et al., ”A Review of molecular recognition technologies for detection of biological threat agents”, Biosensors and Bioelectronics, 15(2000)549.
16. E. Aranda et al., Lett. Appl. Microbiol., 25(1997)186.
17. http://www.jpobd.net
18. P. Belgrader et al., J. Forens. Sci., 43(1998)315;
19. K. Petersen, IVD Technology, Nov./Dec. 1998.
20. Nature Genetics Supplement, 21 1-60, (1999)
21. “Accessing Genetic Information with High-Density DNA Arrays” by Mark Chee, Robert Yang, Earl Hubbell, Anthony Bemo, Xiaohua Huang, David Stem, Jim Winkler, David Lockhart, Macdonald Morris and Stephen Fodor. Science 274 610-614 (1996).
22. “Light-Generated Oligonucleotide Arrays for Rapid DNA Sequence Analysis” by Ann Pease, Dennis Solas, Edward Sullivan, Maureen Cronin, Christopher Holmes and Stephen Fodor. PNAS 99 5022-5026 (1994).
23. “Using oligonucleotide probe arrays to access genetic diversity” by Lipshutz R J, Morris D, Chee M, Hubbell E, Kozal M J, Shah N, Shen N, Yang R, Fodor S P. Biotechniques 19 (3) 442-447 (1995).
24. “Scanometric DNA Array Detection with Nanoparticle Probes” by Andrew Taton, Chad Mirkin and Robert Letsinger. Science 289 1757-1760 (2000).
25. “Rapid Determination of Single Base Mismatch Mutations in DNA Hybrids by Direct Electric Field Control” by Ronald Sosnowski, Eugene Tu, William Butler, James O'Connell and Michael Heller. PNAS 94 1119-1123 (1997).
26. “Monitoring gene expression using DNA microarrays” by Harrington C A, Rosenow C, Retief. J. Curr Opin Microbiol. 3 (3) 285-291 (2000).
27. “Rapid, High Fidelity Analysis of Simple Sequence Repeats on an Electronically Active DNA Microchip” by Ray Radtkey, Lana Feng, M. Muralhidar, Melanie Duhon, David Cantor, Deborah DiPierro, Sylvia Fallon, Eugene Tu, Kevin McElfresh, Michael Nerenberg and Ron Sosnowski. Nucleic Acids Research 28 (7) i-vi (2000).
28. “Single Nucleotide Polymorphic Discrimination by and Electronic Dot Blot Assay on Semiconductor Microchips” by Patrick Gilles, David Wu, Charles Foster, Patrick Dillon and Stephen Chanock. Nature Biotechnology 17 365-370 (1999).
29. http://www.idahotech.com/products/index.html
30. P. Begrader et al., J. Forens. Sci., 43(1998)315;also http://www.llnl.gov/str/Milan.html.
31. M. S. Ibrahim et al., Anal. Chem., 70(1998)2013.
32. K. Petersen, IVD Technology, Nov./Dec. 1998.
33. Clinical Laboratory News, February, 1998.
34. L. A. Christel, K. Petersen, W. McMillan and M. A. Northrup, Trans. ASME, 121(1999)22.
35. Gomez-Navarro, C.; Moreno-Herrero, F.; de Pablo, P. J.; Colchero, J.; Gomez-Herrero, J.; Baro, A. M. Proc. Nat. Acad. Sci. U.S.A. 2002, 99, 8484.
36. Bockrath, M.; Markovic, N.; Shepard, A.; Tinkham, M.; Gurevich, L.; Kouwehoven, L. P.; Wu, M. W.; Sohn, L. L. Nano Lett. 2002, 2, 187.
37. Richter, J. Physica E 2003, 16, 157.
38. Braun, E.; Eichen, Y.; Sivan, U.; Ben-Yoseph, G. Nature 1998,
39. Seidel, R.; Mertig, M.; Pompe, W. Surf. Interface Anal. 2002, 33, 151.
40. Richter, J.; Mertig, M.; Pompe, W.; Monch, I.; Schackert, H. K. Appl. Phys. Lett. 2001, 78(4), 536.
41. Keren, K.; Berman, R. S.; Buchstab, E.; Sivan, U.; Braun, E. Science 2003, 302, 1380.
42. Eichen, Y.; Sivan, U.; Braun, E. PCT WO0025136 1999.
43. J. Wang, Analytica Chim. Acta, 500(2003)247.
44. Mirkin, C. A.; Letsinger, R. L.; Mucic, R. C.; Storhoff, J. J. Nature 1996, 382, 607.
45. Mucic, R. C.; Storhoff, J. J.; Mirkin, C. A.; Letsinger, R. L. “DNA-Directed Synthesis of Binary Nanoparticle Network Materials,” J. Am. Chem. Soc., 1998, 120, 12674-12675.
46. Storhoff, J. J.; Lazaorides, A. A.; Mucic, R. C.; Mirkin, C. A.; Letsinger, R. L.; Schatz, G. C.; “What Controls the Optical Properties of DNA-Linked Gold Nanoparticle Assemblies?” J. Am Chem. Soc., 2000, 122, 4640-4650.
47. Elghanian, R.; Storhoff, J. J.; Mucic, R. C.; Letsinger, R. L.; Mirkin, C. A. “Selective Colorimetric Detection of Polynucleotides Based on the Distance-Dependent Optical Properties of Gold Nanoparticles,” Science, 1997, 277, 1078-1080.
48. S. Park, T. A. Taton, C. A. Mirkin, Science 295 (2002) 1.
49. Effenhauser, C. S.: Integrated chip-based microcolumn separation systems. in: Manz, A. Becker, H.: Microsystem technology in chemistry and life sciences, Topics in Current Chemistry, SpringerVerlag, Heidelberg, 1998.
50. A. H. Foster, M. kriha,. P. D. Swanson. T. C. Young and D. E. Ackley, Biosenstors and Bioelectronics 16(2001)187.
51. C, F, Edman, D. E. Raymond, D. J. Wu, E. Tu, R.G. Sosnowski, W. F. Butler, M. Nerenberg and M. J. Heller, Nucl. Acids Res., 24(1997)25.
52. P. Swanson, R. Gelbart, E. Atlas, Li Yang, T. Grogan, W. F. Butler, D. E. Ackley and E. Sheldon, Sensors and Actuators B, 64(2000)22.
53. P. N. Gilles, D. J. Wu, C. B. Foster, P. J. Dillon and S. J. Chanock, Nature Biotechnol., 17(1999)365.
54. “Strand Displacement Amplification—An Isothermal, In Vitro DNA Amplification Technique” by Terrance Walker, Melinda Fraiser, James Schram, Michael Little, James Nadeau and Douglas Malinowski. Nucleic Acids Research 20 (7) 1691-1696 (1992).
55. “Multiplex Strand Displacement Amplification (SDA) and Detection of DNA Sequences from Mycobacterium tuberculosis and Other Mycobacteria” by Terrance Walker, James Nadeau, Patricia Spears, James Schram, Colleen Nycz and Daryl Shank. Nucleic Acids Research 22 (13) 2670-2677 (1994).
56. “Impedance Spectroscopy”, J R Macdonald, ed., John Wiley, 1987. Sect. 2.2.3.4.

1. A method for the electronic detection of hybridized targets comprising the steps of:
providing an electronic microarray having specific probes associated with two or more microarray locations,
electronically accumulate the target on a particular electrode site at the micro-electrode array,
sequential electronic hybridization of oligonucleotide labeled conductive particles on the target, and
monitoring the electrochemical AC impedance changes at the electrode site.

2. The method of claim 1 wherein the target is a genomic target.

3. The method of claim 2 wherein the genomic target is a nucleic acid.

4. The method of claim 3 wherein the nucleic acid is DNA.

5. The method of claim 3 wherein the nucleic acid is RNA.

6. The method of claim 1 wherein the particles are nanoparticles.

7. The method of claim 1 further including the step of electroplating over the DNA target.

8. The method of claim 4 wherein the target serves as the metallization template.

9. The method of claim 1 wherein the conductive particles are metallic particles.

10. The method of claim 9 wherein the metallic particles are gold.

11. The method of claim 9 wherein the metallic particles are silver.

12. The method of claim 9 wherein the metallic particles are palladium.
